(19) United States
(12) Patent Application Publication
(10) Pub. No.: US 2021/0059566 A1
(43) Pub. Date: Mar. 4, 2021

(54) Step Analysis Device

(71) Applicants: Raphi Blustain, Tel Aviv (IL); Shachar Holander, Tel Aviv (IL)

(72) Inventors: Raphi Blustain, Tel Aviv (IL); Shachar Holander, Tel Aviv (IL)

(21) Appl. No.: 16/961,032

(22) PCT Filed: Jan. 14, 2019
(86) PCT No.: PCT/IL2019/050052

§ 371 Date: Jul. 9, 2020

Related U.S. Application Data
(60) Provisional application No. 62/617,338, filed on Jan. 15, 2018.

(51) Int. Cl. A61B 5/11; A61B 5/00

(52) U.S. Cl. CPC A61B 5/112; A61B 2562/0219; A61B 5/6807; A61B 5/746

(57) ABSTRACT

A step analysis device comprises at least one motion sensor for measuring instantaneous direction-specific movement of a user made during an ambulatory activity; at least one deviation indicator for alerting the user during the ambulatory activity upon determination of deviative motion; and a processor for processing signals generated by the at least one motion sensor that are indicative of the instantaneous direction-specific movement, and for comparing the instantaneous direction-specific movement with a normative direction-specific movement. The processor is operable to command the at least one deviation indicator to generate a predetermined biofeedback alert upon determining a deviation greater than a predetermined threshold level between the instantaneous direction-specific movement and the normative direction-specific movement.

### FIELD OF THE INVENTION

The present invention relates to the field of health care. More particularly, the invention relates to a step analysis device that is suitable for correcting bad walking such as in-toeing or out-toeing.

### BACKGROUND OF THE INVENTION

In a normal walking style, people walk with their toes pointing substantially forwardly. A deviation from the normal walking style, such as an in-toeing gait or an out-toeing gait, can cause damage to one's ankles, feet, knees, hips and back.

Often people who deviate from the normal walking style do so due to poor walking habits, imbalance of muscle strength or heredity. Many times, walking can be corrected by acquiring good and healthy walking habits.

It is an object of the present invention to provide a compact step analysis device which helps a user to correct bad walking habits.

It is an additional object of the present invention to provide a self-contained step analysis device that is capable of both monitoring a foot position during an ambulatory activity and independently indicating when the foot position needs to be corrected.

Other objects and advantages of this invention will become apparent as the description proceeds.

### SUMMARY OF THE INVENTION

A step analysis device comprises at least one motion sensor for measuring instantaneous direction-specific movement of a user made during an ambulatory activity; at least one deviation indicator for alerting the user during the ambulatory activity upon determination of deviative motion; and a processor for processing signals generated by the at least one motion sensor that are indicative of the instantaneous direction-specific movement, and for comparing the instantaneous direction-specific movement with a normative direction-specific movement, wherein the processor is operable to command the at least one deviation indicator to generate a predetermined biofeedback alert upon determining a deviation greater than a predetermined threshold level between the instantaneous direction-specific movement and the normative direction-specific movement.

Even though generation of the biofeedback alert is foot-specific, a step analysis device may be mounted in each shoe of the user such that corresponding processors of first and second step analysis devices are synchronized in a master-slave arrangement to facilitate comparison of corresponding measured signals.

The invention is also directed to a shoe that comprises the step analysis device.

In one aspect, the at least one motion sensor, the at least one deviation indicator, and the processor are housed in a monolithic enclosure that is coupleable with a shoe part of a single shoe.

In one aspect, the processor is operable in inactive, measuring and calibrating modes, and is operable to generate the normative direction-specific movement in the calibrating mode.

In one aspect, the step analysis device is a walk correction device, and the processor is operable to command generation of the biofeedback alert upon determining an angular difference greater than the predetermined threshold between an instantaneous foot orientation and a normative forward direction of movement.

In one aspect, the processor is operable to command generation of the biofeedback alert upon determining a deviation between an instantaneous running pattern and a reference running pattern.

### BRIEF DESCRIPTION OF THE DRAWINGS

In the drawings:

FIG. 1 is a block diagram of an embodiment of a step analysis device, when schematically illustrated from above with respect to a shoe part within which it is mounted;

FIG. 2 is a block diagram of an embodiment of a step analysis device;

FIG. 3 is a flow chart for determining whether walk correction is necessary; and

FIG. 4 is a partial block diagram of another embodiment of a step analysis device, together with other devices with which it is in wireless communication.

### DETAILED DESCRIPTION OF THE INVENTION

The step analysis device of the present invention is adapted to assist in correcting a deviative motion that is made while walking, jogging or running (hereinafter referred to as an “ambulatory activity”), for example caused by poor walking habits. Each placement of the foot on an underlying surface will be referred to a “step”. The device is mounted within a single shoe of a user, or to any suitable part associated with the single shoe, and is capable of detecting the deviative motion and of alerting the user that a correction is needed. One implementation of the step analysis device is a walk correcting device that is configured to remind a user to walk in a normal walking style whereby the toes point substantially forwardly. Since the step analysis device is mounted in a single shoe, the alert is foot-specific, and does not take into consideration the foot position of the other foot.

The device comprises at least one motion sensor, such as an accelerometer, for measuring the user's instantaneous foot position; at least one deviation indicator, such as an LED, a vibration element or an enunciator, for alerting the user when a deviative motion is detected; and a processor for processing measurements from each motion sensor to enable generation of an alert. The device can be mounted on an insole or on any other shoe part worn on a user's foot. In some embodiments, at least one orientation sensor, such as a digital compass, can be used for determining the direction in which the user's foot points.

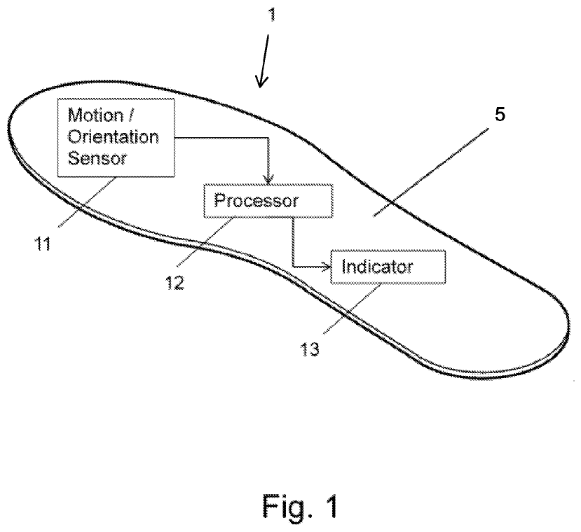

FIG. 1 schematically illustrates an embodiment of a step analysis device 1, which is mounted on a shoe part 5 and configured to assist in walk correction. A motion/orientation sensor 11 measures the instantaneous foot orientation when walking. One or more are transmitted to a processor 12. Processor 12 processes one or more signals indicative of the measurements and compares this measured orientation to a normative forward direction of movement. If the difference between the instantaneous foot orientation and the normative forward direction of movement is greater than a predetermined threshold or a predetermined angular range (such as greater than a few degrees, e.g. 10-15 degrees, when a foot is pointing inwardly or outwardly instead of pointing straight ahead), processor 12 activates deviation indicator 13 to alert the user as to the deviation. The components of device 1 may be distributed, or alternatively may be mounted monolithically.

A user can personalize the device, for instance by determining the type and duration of alerts, and by determining the device sensitivity (i.e. the minimum difference between measurements made by the processor).

In some embodiments, the device may collect data regarding the walking habits and behavior of a user (e.g., level of a rotational variation of the lower extremity when the foot points toward the midline or away from the midline during gait, and intervals between alerts). The collected data can be used for several applications, e.g., to show the progress of the user for gaining healthy walking habits.

Although the description relates to forward movement, the step analysis device is likewise capable of determining deviative motion during rearward or lateral movement.

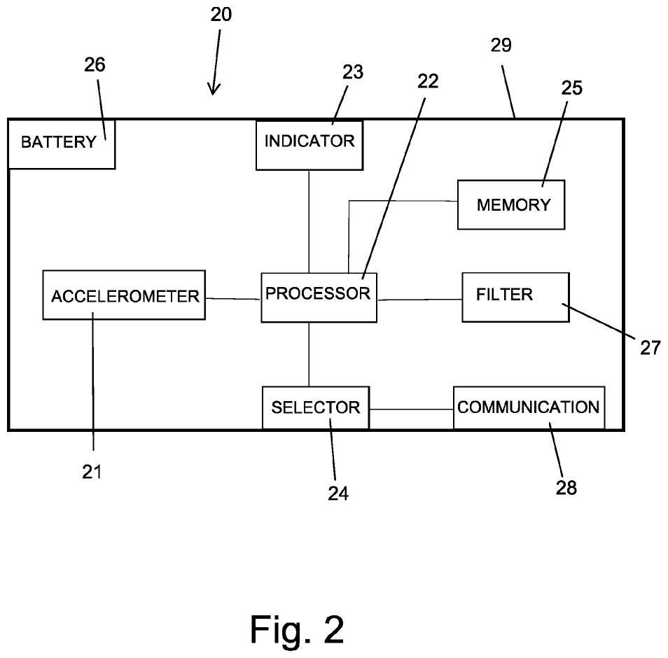

FIG. 2 schematically illustrates a walk correcting device 20, according to another embodiment. Walk correcting device 20 comprises a three-axis accelerometer 21 for determining the instantaneous foot orientation, a processor 22 in data communication with accelerometer 21, a deviation indicator 23 in data communication with processor 22, and a battery 26, or other suitable power source together with battery charging and power management circuitry, for powering accelerometer 21, processor 22 and deviation indicator 23. Processor 23 is generally configured with a selector 24, for selecting a mode of operation, whether an inactive mode, a measuring mode for measuring the instantaneous foot orientation, or a calibrating mode. Selector 24 may be activated by an input device, such as a small pressable switch that is accessible to the user, or by means of a remote control device such as one configured with a user interface that is in wireless data communication with a suitable communication device 28, such a short-range transceiver.

Accelerometer 21 is adapted to continuously, periodically or intermittently measure the instantaneous foot orientation of a user during an ambulatory activity. Generated voltage, generally resulting from stressed microscopic crystal structures, is indicative of a velocity vector and thus of the foot orientation. If the angular difference between the instantaneous foot orientation and the normative forward direction of movement is greater than the predetermined threshold, processor 22 activates deviation indicator 23, causing the alert to be provided to the user during the course of the ambulatory activity. Deviation indicator 23 may be embodied by various components such as a LED lamp that generates a perceptible beam, an enunciator that vocalizes a prerecorded message or a buzzing sound, or a vibration element.

A filter 27, usually a low-pass filter, which is in data communication with processor 22, or may be a module of the processor, may be used to improve the calculation output of the processor in the measuring mode and calibrating mode. In a normal walking style, each leg is involved in a different type of motion, such as rocking with the heel, lifting the foot from the floor, extending the knee and hip, flexing the knee and hip, and striking the floor with the foot. Each different type of motion is involved in helping to propel the body forwardly and is associated with varying magnitudes of force applied to the floor, and, through interaction with accelerometer 21, with varying levels of vibrations. Filter 27 is configured to filter out those measurements of the instantaneous foot orientation performed during leg motions which are associated with excessive noise, for example due to shock absorption of an extended knee or lowering the foot, with respect to the floor. Filter 27 may be operable to filter out the signals generated by accelerometer 21 before being transmitted to processor 22 when the corresponding foot displacement in the z-axis is greater than a predetermined threshold or when the corresponding signal to noise ratio is less than a predetermined threshold. For example, filter 27 may attenuate signals greater than 100 Hz. A typical filter may be one with a sample rate of 200 Hz and a cutoff frequency of 60 Hz.

Data measured by accelerometer 21 or processed by processor 22 may be stored in memory device 25, in order to be retrieved at a later time for future analysis.

All of these components may be housed in a monolithic enclosure 29, which is generally rigid, to minimize enclosure movement and to optimize measurement accuracy. The thin device enclosure 29, which generally has the size of a watch, is coupled to a suitable shoe part, such as an insole, outsole, tongue or heel. If so desired, enclosure 29 may be mounted within a dedicated cavity provided with an insole.

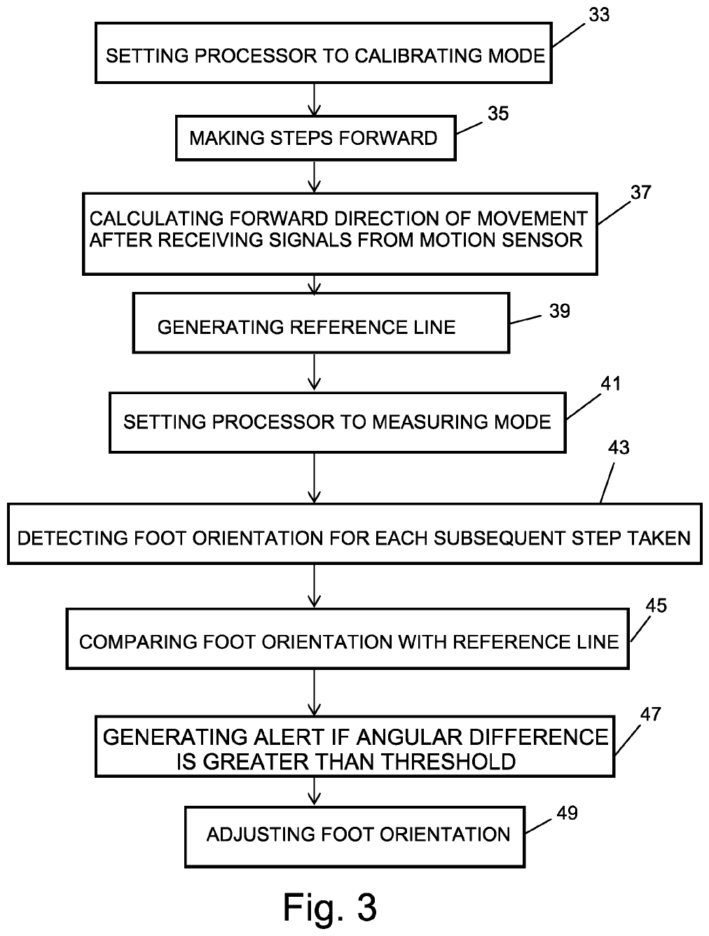

FIG. 3 illustrates a flow chart for determining whether walk correction is necessary. The description relates to forward motion of the user, but it is likewise applicable to rearward motion.

Whenever required, for example prior to first time use, the processor is set to calibrating mode in step 33 and the user then makes a few steps forward in step 35. The motion sensor detects the foot orientation for each step, and the processor, after processing the signals generated by the motion sensor that define a corresponding force vector , calculates the normative forward direction of movement in step 37 by combining the corresponding force vectors in accordance with predetermined instructions. Based on the calculated normative forward direction of movement, the processor generates an imaginary reference line in step 39 that corresponds to the calculated normative forward direction of movement, along which the user is expected to continue walking if correct walking habits are exhibited.

After the processor is subsequently set to the measuring mode in step 41, the user naturally and uninhibitedly walks in step 43 while the motion sensor detects the instantaneous foot orientation for each step. The processor processes the signals generated by the motion sensor that are indicative of the instantaneous foot orientation, and compares the instantaneous foot orientation with the normative forward direction of movement in step 45. If the angular difference between the instantaneous foot orientation and the normative forward direction of movement is greater than a predetermined threshold, the processor commands the deviation indicator to generate a predetermined alert signal in step 47. The alert urges the user to consciously or instinctively adjust the foot orientation in step 49. If the alert signal is repeatedly generated after each attempt to adjust the foot orientation, the user realizes that the device enclosure has moved and that calibration has to be performed once again.

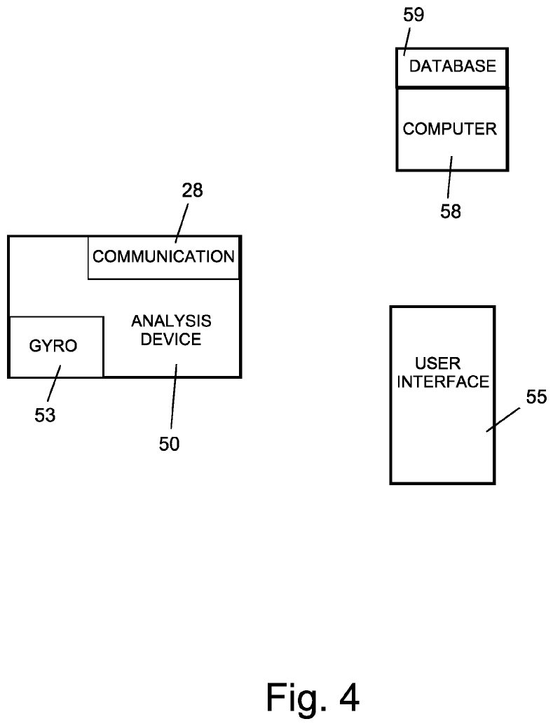

FIG. 4 schematically illustrates step analysis device 50. Step analysis device 50 is configured similarly to device 20 of FIG. 2, but with the addition of a gyroscope 53, for measuring three-dimensional spatial motion during ambulatory activities, in addition to, or in place of, the accelerometer, such as in conjunction with a MEMS.

By virtue of the ability to accurately and reliably measure spatial motion, step analysis device 50 is capable of determining whether the user is deviating from the normative forward movement while running or jogging.

Since running motion is different than walking motion, a different calibration procedure is needed. In contrast to walking, both legs of a runner become separated from the ground and sequentially contact with the ground at the center of the foot in a landing stage. Also, the impact onto the ground while running is approximately three times the weight of the body, as opposed to being approximately 90% of body weight while walking. Additionally, both legs of a runner follow a same line while running and have a relatively long stride.

Accordingly, after the calibrating mode is set, the user wearing step analysis device 50 is instructed to perform a running motion. During the running motion, the one or more motion sensors is able to detect the spatial orientation of various lower body parts such as the knee, ankle and foot for each step, and to distinguish between motions of a running cycle. The processor receives the signals generated by the one or more motion sensors that define a corresponding force vector and calculates the normative forward movement by combining the corresponding force vectors in accordance with predetermined instructions. The calculated normative forward movement is representative of a reference pattern to which is compared the running style of the user which is detected in the subsequent measuring mode. The processor generates a biofeedback alert by means of the deviation indicator upon detecting a deviation from the reference running pattern.

Step analysis device 50 is able to communicate wirelessly with a computerized device 58, such as mobile device, laptop computer and server, so that the acquired and analyzed data is able to be stored in an associated database 59 for later analysis.

A dedicated application may be running on the processor of step analysis device 50, and a user interface 55 may interface with the application by means of communication device 28. User interface 55 may be used for initiating one of the modes of operation, defining various settings, such as those of the deviation indicator and one or more motion sensors, adding an identifier to an analysis session, and uploading a desired reference running pattern from database 59 to the processor.

A reference running pattern may focus on a single motion of a running pattern that needs to be improved or is desired to be emulated, such as the height a foot is lifted from the ground during a take-off stage, the length of a running stride, or the way a foot contacts the ground and becomes separated from the ground, such that an alert will be generated only when the single motion is found to be deviative. Alternatively, all motions of the reference running pattern may be the basis of comparison with the instantaneous running pattern of the user. A score may be calculated from a comparison between corresponding motions of the reference and instantaneous running patterns, according to stored instructions, and an alert will be generated if the score is less than a predetermined threshold.

A reference running pattern may also be uploaded by a training specialist or physician in order to prevent injuries while running. Such a reference running pattern may define restrictions of the user, so that the biofeedback alert will be generated whenever a set user-specific maximum value is exceeded. Exemplary restrictions include a maximum running speed or acceleration.

Step analysis device 50 may also be used as a diagnostic tool, to analyze the performance of athletes during the course of an athletic activity, or to collect statistics or other pre-defined features related to a running session. The data received from step analysis device 50 can be used to compare the athlete's performance at a specific time or event of the athletic activity with stored data, so that the athlete's performance progress can be monitored.

It will be appreciated that any of the aforementioned features of step analysis device 50 are also applicable to a walk correction device.

In one embodiment, a step analysis device 50 is mounted in each shoe of the user. Even though each step analysis device 50 is foot-specific, the two identical step analysis devices may be in wireless communication with each other by the corresponding communication devices 28, allowing the corresponding processors to be synchronized in a master-slave arrangement to compare the corresponding measured signals. Thus one of the step analysis devices may generate a biofeedback alert if the corresponding foot is found to undergo deviative motion, such as being asymmetrical with respect to the other foot.

While some embodiments of the invention have been described by way of illustration, it will be apparent that the invention can be carried out with many modifications, variations and adaptations, and with the use of numerous equivalents or alternative solutions that are within the scope of persons skilled in the art, without exceeding the scope of the claims.

1-8. (canceled)

9. A walk correction method, comprising:
a) measuring instantaneous direction-specific movement of a user made during an ambulatory activity by at least one motion sensor;
b) alerting the user during the ambulatory activity upon determination of deviative motion by using at least one deviation indicator; and
c) processing, by a processor, signals generated by the at least one motion sensor that are indicative of the instantaneous direction-specific movement, comparing the instantaneous direction-specific movement with a normative direction-specific movement, and
commanding the at least one deviation indicator to generate a predetermined biofeedback alert upon determining a deviation greater than predetermined threshold level between the instantaneous direction-specific movement and the normative direction-specific movement.

10. The method according to claim 9, wherein the at least one motion sensor, the at least one deviation indicator, and the processor are housed in a monolithic enclosure that is coupleable with a shoe part of a single shoe.

11. The method according to claim 9, wherein the processor is operable in inactive, measuring and calibrating modes, and is operable to generate the normative direction-specific movement in the calibrating mode.

12. The method according to claim 9, wherein the processing comprising commanding generation of the biofeedback alert upon determining an angular difference greater than the predetermined threshold between an instantaneous foot orientation and a normative forward direction of movement.

13. The method according to claim 9, wherein the processor is operable to command generation of the biofeedback alert upon determining a deviation between an instantaneous running pattern and a reference running pattern.

14. The method according to claim 9, wherein the processor is operable to command generation of the biofeedback alert which is foot-specific.

15. The method according to claim 14, which is mountable in each shoe of the user such that corresponding processors of associated with each foot are synchronized in a master-slave arrangement to facilitate comparison of corresponding measured signals.

16. The method according to claim 9, further comprising:
a) setting the processor to calibrating mode;
b) detecting the food orientation for each step the user make; and
c) defining a corresponding force vector and calculating the normative forward direction of movement by combining the corresponding force vectors in accordance with predetermined instructions, wherein based on the calculated normative forward direction of movement, generating an imaginary reference line that corresponds to the calculated normative forward direction of movement, along which the user is expected to continue walking if correct walking habits are exhibited.
